(19) United States
(12) Patent Application Publication
(10) Pub. No.: US 2002/0072804 A1
(43) Pub. Date: Jun. 13, 2002

(54) Combination biological-non-biological material prosthetic implant and method

(76) Inventor: Russell S. Donda, Alachua, FL (US)

Correspondence Address: VAN DYKE & ASSOCIATES, P.A., 1630 HILLCREST STREET, ORLANDO, FL, 32803, US

(21) Appl. No.: 09/957,858

(22) Filed: Sep. 20, 2001

Related U.S. Application Data
(60) Provisional application No. 60/234,014, filed on Sep. 20, 2000.

(51) Int. Cl. A61F 2/28; A61F 2/08

(52) U.S. Cl. 623/23.26; 623/13.14; 623/23.63

(57) ABSTRACT

This invention provides a method and means for securing a tissue to a bone or non-tissue prosthesis by affixing a collagenous material to said bone or non-tissue prosthesis, and affixing said tissue to said collagenous material.

### CROSS-REFERENCE TO RELATED APPLICATION

This application claims the benefit under 35 USC §119(e) of Provisional Application No: 60/234,014 filed Sep. 20, 2000.

### BACKGROUND OF THE INVENTION

1. Field of the Invention

This invention relates to the area of prosthetic implants and methods for securing biological tissues to such implants.

2. Background

In recent years, it has become increasingly common for people and animals to receive prosthetic implants composed substantially of non-biological materials. As a result, there has been a developing recognition of the problems that occur at the interface of biological tissue with the non-biological prosthesis.

Thus, for example, in U.S. Pat. No. 4,599,084, there is disclosed a method of using biological tissue to promote even bone growth by interposing a barrier layer of glutaraldehyde-fixed pericardial tissue at the interface of resected bone and an implant, in the hope of minimizing the deleterious effects of spurious bone growth adjacent the implant. Thus, the '084 patent is primarily directed to provision of a protective device for an implant.

In U.S. Pat. No. 4,778,473, a prosthesis interface surface and method of implanting was disclosed, wherein a prosthetic implant surface intended for long term bone fixation is made of a porous metal or ceramic, or coating the surface of the implant with a porous metal or ceramic.

In U.S. Pat. No. 3,973,277, a method of attaching fibrous connective tissue to bone was disclosed, wherein a natural or artificial ligament or tendon is connected to bone by providing a tapered plug at the terminus of the connective tissue which is placed in a corresponding resection in bone to which the fibrous connective tissue is to be secured.

In U.S. Pat. No. 4,772,288, a method was disclosed for making an implantable ligament or tendon prosthesis from collagen wherein collagen fibers could be composited with tissue fibers or synthetic fibers.

In U.S. Pat. No. 5,157,111, a method of bonding collagen fibers to synthetic fibers, such as dacron, by covalently bonding the collagen to chemically activated polymer fibers. Essentially, the disclosure relates to a method of using a fiber such as dacron as a replacement ligament whereby the dacron fibers are bonded to collagen to assist in fibroblast growth on the dacron fibers.

In U.S. Pat. No. 5,171,273, a synthetic collagen orthopedic structure was disclosed wherein a tendon graft was made from cross-linked and non-cross-linked collagen fibers.

In U.S. Pat. No. 5,002,583, an implant was disclosed comprising a synthetic material from which collagen fibers are made to protrude.

In U.S. Pat. No. 5,925,078, a method and apparatus for joining collagen-containing materials was disclosed wherein collagen fibril ends are contacted and thermally fused to each other.

While technologies such as those described above may have applications in the joining of prosthetic implants of non-biological material to biological implant sites, none of these identified documents solve the problem of achieving increased stability and biocompatibility of the mating surfaces joining the body's tissues to a prosthetic implant in the efficient and stable manner disclosed herein.

Accordingly, it is an object of this invention to provide a method and implant for effective combining of autograft, allograft or xenograft tissues with a prosthetic implant which allows a patient's tissue to mate with the autograft, allograft or xenograft tissue combined with said prosthetic implant.

A further object of this invention is to provide a means to effect attachment of a patient's soft tissue, flexible tissue, or fibrous tissue, such as ligament or tendon, to a non-tissue prosthesis by mating said prosthesis with a collagen implant, thus providing a point of attachment of tissue to said non-tissue prosthetic implant.

A further object of this invention is to provide a collagen implant lined, packed or filled with an osteogenic material or bone paste.

A further object of this invention is to provide a non-tissue prosthetic implant to which is secured a collagen implant.

A further object of this invention is to provide a method for affixing a tissue to a bone or non-tissue prosthetic implant, which comprises affixing a collagen implant to said bone or non-tissue prosthetic implant, and affixing said tissue to said collagen implant.

### SUMMARY OF THE INVENTION

This invention provides a method and means for securing a tissue to a bone or non-tissue prosthesis by affixing a collagenous material to said bone or non-tissue prosthesis, and affixing said tissue to said collagenous material.

### DETAILED DESCRIPTION OF THE PREFERRED EMBODIMENTS OF THE INVENTION

According to the present invention, fixation of tissues to bone or to non-tissue prosthetic implants is facilitated by fixing a collagenous material to said bone or non-tissue prosthetic implant, and then fixing said tissue to said collagenous material. Naturally, those skilled in the art will appreciate that in certain circumstances, it might be preferred to first affix said collagenous material to said tissue, and then affix said collagenous material to said bone or said non-tissue prosthesis. However, for the purposes of this patent disclosure, the emphasis is placed on first affixing the collagenous implant to the bone or non-tissue prosthesis to which said tissue is intended to be affixed.

According to this invention, any of a number of different collagenous materials are desirable. Included within this definition are skin, pericardium, peritoneum, demineralized bone, fascia or the like. The collagenous material may preferably be provided in the form of a continuous ring or the like, such that the collagenous material may be fitted around the bone or non-tissue implant. The collagenous material may be screwed, stapled or otherwise mechanically affixed to the bone or non-tissue implant. The collagenous material may be chemically bonded to the prosthetic material according to methods known in the art. Furthermore, the collagenous material may be chemically treated to modify the physical and chemical properties of said implant.

In a first embodiment, an implant composed of bone, synthetic polymer, metal or combinations thereof is affixed in the intramedullary canal of the femur. The implant may comprise the head of the femur for insertion into the acetabulum. Ligaments and tendons are affixed to a ring of demineralized bone affixed to the exterior of the bone implant, the non-tissue prosthesis or to the shaft of the femur, and ligaments or tendons are affixed to the demineralized bone.

In a second embodiment, the collagenous material is a portion of fascia.

In a third embodiment, the collagenous material is skin.

In a fourth embodiment, the collagenous material has been cross-linked with glutaraldehyde or another tanning chemical.

In a fifth embodiment, the collagenous material is affixed to a prosthesis in the shoulder, the wrist, the arm, the hand, the leg, the spine, the foot, or the jaw.

In a sixth embodiment, the collagenous material is affixed to a prosthesis via chemical means or mechanical means.

In order to promote bone growth, the collagenous material according to this invention may be impregnated, coated, infused, or filled with an osteogenic material, such demineralized bone matrix, bone morphogenetic proteins, growth factors and the like. If other types of tissue growth is desired, those skilled in the art will appreciate from this disclosure that appropriate alternate growth factors and tissue growth stimulants should be selected.

What is claimed is:

1. A method for effective combining of autograft, allograft or xenograft tissues with a non-tissue prosthetic implant which allows a patient's tissue to mate with the autograft, allograft or xenograft tissue combined with said prosthetic implant.

2. A means to effect attachment of a patient's soft tissue, flexible tissue, or fibrous tissue, such as ligament or tendon, to a non-tissue prosthetic implant which comprises mating said prosthetic implant with a collagen implant, thus providing a point of attachment of tissue to said non-tissue prosthetic implant.

3. A collagen implant lined, packed or filled with an osteogenic material, hardening cement, or bone paste.

4. A collagen implant mating with the surface of a prosthetic implant at the point where an osteogenic material has been placed, comprising demineralized bone matrix, bone morphogenic proteins, bone cell growth factors, and combinations thereof.

5. A non-tissue prosthetic implant to which is secured a collagen implant.

6. A method for affixing a tissue to a bone or non-tissue prosthetic implant, which comprises affixing a collagen implant to said bone or non-tissue prosthetic implant, and affixing said tissue to said collagen implant.

7. A method for securing a tissue to a bone or a non-tissue prosthesis by affixing a collagenous material to said bone or non-tissue prosthesis, and affixing said tissue to said collagenous material.

8. A method for fixation of tissues to bone or to non-tissue prosthetic implants comprising fixing a collagenous material to said bone or non-tissue prosthetic implant, and then fixing said tissue to said collagenous material.

9. A method for fixation of tissues to bone or to non-tissue prosthetic implants comprising first affixing said collagenous material to said tissue, and then affixing said collagenous material to said bone or said non-tissue prosthesis.

10. A method for fixation of tissues to bone or to non-tissue prosthetic implants comprising first affixing said collagenous material to said bone or said non-tissue prosthesis, and then affixing said collagenous material to said tissue. [but see note on page 6 in left margin]

11. The method according to claim 1 wherein said collagenous material is selected from the group consisting of skin, pericardium, peritoneum, demineralized bone, fascia and combinations thereof.

12. The method according to claim 9 wherein said collagenous material is provided in the form of a continuous ring, such that the collagenous material may be fitted around the bone or non-tissue implant.

13. The method according to claim 9 wherein said collagenous material is screwed, stapled or chemically affixed to the bone or non-tissue implant.

14. The method according to claim 9 wherein said collagenous material is chemically bonded to the prosthetic material.

15. The method according to claim 9 wherein said collagenous material is chemically treated to modify the physical and chemical properties of said implant.

16. The method according to claim 9 comprising affixing an implant composed of bone, synthetic polymer, metal or combinations thereof in the intramedullary canal of the femur, wherein said implant comprises the head of the femur for insertion into the acetabulum, and fixing ligaments or tendons to a ring of demineralized bone, skin or fascia affixed to the exterior of the bone implant, the non-tissue prosthesis or to the shaft of the femur.

17. The method according to claim 14 wherein the collagenous material has been cross-linked with glutaraldehyde.

18. The method according to claim 1 wherein the collagenous material is affixed to a prosthesis in the shoulder, the wrist, the arm, the hand, the leg, the spine, the foot, or the jaw.

19. The method according to claim 1 wherein the collagenous material is affixed to a prosthesis via chemical means or mechanical means.
